(19) United States
(12) Patent Application Publication
(10) Pub. No.: US 2007/0219460 A1
(43) Pub. Date: Sep. 20, 2007

(54) ASPIRATION NEEDLES

(76) Inventor: Alec S. Goldenberg, New York, NY (US)

Correspondence Address: DARBY & DARBY P.C., P.O. BOX 770, Church Street Station, New York, NY, 10008-0770, US

(21) Appl. No.: 11/685,543

(22) Filed: Mar. 13, 2007

Related U.S. Application Data
(60) Provisional application No. 60/783,066, filed on Mar. 15, 2006.
(60) Provisional application No. 60/787,731, filed on Mar. 29, 2006.

(51) Int. Cl. A61B 10/00

(52) U.S. Cl. 600/566; 600/562; 600/565; 600/567

(57) ABSTRACT

According to one aspect of the present invention, an aspirating needle for collecting a specimen includes an elongated body that includes a hollow cannula portion that is open at both a proximal end and a distal end for placement at a specimen site to collect and permit aspiration of the specimen from the specimen site. The cannula portion has a plurality of side openings formed along a length of the cannula portion and proximate the open distal end for providing entrances into a hollow interior of the cannula portion, wherein only one side opening is located in a single transverse plane taken across the cannula portion at a right angle to a longitudinal axis of the cannula portion.

### CROSS-REFERENCE TO RELATED APPLICATION

The present application claims the benefit of U.S. patent application Nos. 60/783,066, filed Mar. 15, 2006 and 60/787,731, filed Mar. 29, 2006, both of which are hereby incorporated by reference in their entirety.

### TECHNICAL FIELD

The present invention relates to an instrument, typically known as a needle or cannula that is used to gather a sample from a site using aspiration and more particularly, relates to an aspiration needle for gathering tissue from living persons or animals for pathological study and includes an improved structure for collecting a fluid sample of bone marrow.

### BACKGROUND

For various medical reasons, such as diagnostic tests or the like, it is often necessary for a physician to obtain a sample of a specific tissue from a patient Often, a biopsy (sample) is required from a rigid structure, such as a bone or bone marrow specimen. Bone marrow biopsies are typically recovered with significant portions of their internal bony structure intact which allows the pathologist to provide interpretations regarding bone marrow cellularity or infiltration with abnormal cells.

A bone marrow sampling procedure usually includes both the collection of a core biopsy using a bone marrow biopsy needle and a fluid sample of bone marrow using an aspiration needle. The two specimens provide complementary information that is relevant for the evaluation of a variety of malignant and nonmalignant hematologic processes. The bone marrow aspiration provides a liquid sample of suspended hematopoietic progenitor cells, stromal cells, and trabecular bone fragments that can be processed for flow cytometric analysis of the bone marrow content, for cytogenetic studies, as well as for the preparation of smears for detailed morphologic evaluation of the progenitor cell morphology. The core biopsy provides accurate information regarding the status of the supporting bone, the cellularity of the bone marrow sample, and the identification of extrinsic cells as seen when the bone marrow is infiltrated with lymphoma or carcinoma.

Evaluation of the content and morphology of bone marrow tissue is most commonly accomplished by studying both liquid aspirate samples and core biopsy specimens of the bone marrow. Bone marrow aspiration and bone marrow biopsy needles are used to recover the fluid and solid bone marrow specimens, respectively. Occasionally, bone marrow biopsy needles are used to aspirate fluid samples of bone marrow.

Bone marrow biopsy needles are designed to recover relatively solid cores of tissue for accurate assessment of bone marrow cellularity, possible marrow infiltration with neoplastic cells, and the content and structure of bone marrow supporting cells and tissues. A variety of bone marrow biopsy needles are available including newer types which incorporate specimen-capturing mechanisms. Bone marrow biopsy designs have focused on maximizing non-disruptive cutting and recovery of intact tissue biopsy specimens.

Bone marrow aspiration needles have been designed to maximize the recovery of fluid and cellular bone marrow material from the bone marrow space. Generally, these needles are of simple design and include a hollow cannula with a beveled sharpened penetrating tip, a stylet and a handle. In simplistic terms, most aspiration needles are simple tubes with sharp tips for penetrating through the bony cortex. They incorporate a stylet to strengthen and stabilize the cannula and eliminate the possibility of material entering into the tube during the process of bone penetration which might result in obstruction of the tube and inability to aspirate a sample.

The objective of a bone marrow aspiration procedure is to recover small fragments of bone marrow tissue referred to as spicules and not to simply remove blood from the bone marrow space. Spicules are fragments of bone marrow material composed of cellular bone marrow precursor cells which are embedded in fragments of stromal cells and supporting structures. Therefore, the process of bone marrow aspiration actually attempts to recover portions of tissue embedded in a bloody fluid environment. In that sense, it may be more appropriate to view the bone marrow aspiration procedure as a sampling of an nonhomogenous tissue rather than the aspiration of a relatively simple homogenous fluid material, such as blood.

Simple tubes are appropriate for sampling, recovering and transferring less complex fluids as their flow dynamics are more consistent with an ideal fluid. As a result, simple tubes and needles are appropriate for the recovery and sampling of plasma and blood. However, more sophisticated tube designs can be more appropriate for the sampling, recovery and transferring of more complex fluids composed of nonhomogeneous mixtures of plasma and tissue fragments/components. As bone marrow material is a complex fluid/tissue, its sampling with simple aspiration needles may not be efficient and may be improved by designs that account for the non-homogeneous nature of bone marrow material.

The efficient removal of a fluid from a defined space requires equilibration of pressures to eliminate the possibility of producing a negative internal pressure counterproductive to the release of fluid. Recently, vented bone marrow aspiration needles have been proposed to facilitate pressure equilibration and maximize flow of non-homogeneous aspirated bone marrow tissue.

Efficient recovery of aspirated material requires maximal flow of all bone marrow components through the aspiration tube. Flow depends not only on the diameter of the tube or cannula but the configuration and cross-sectional area available for material to enter the tube. The total cross-sectional diameter of entry points can have a more significant effect on the flow of heterogeneous fluids than simple homogenous fluid samples. Therefore increasing the total cross-sectional diameter of entry points will increase flow only to be limited by the diameter of the tube. Increasing the total entry point cross-sectional diameter is however limited by the structural confines of the cannula since penetrating the walls of the cannula with multiple holes may lead to structural instability. Therefore, maximizing the total entry hole cross-sectional diameter while maintaining structural integrity should facilitate flow of nonhomogeneous bone marrow aspirate samples while providing a safe and usable aspiration device. Achieving these results in relatively small diameter cannulas or tubes is a challenging task.

### SUMMARY

According to one aspect of the present invention, an aspirating needle for collecting a specimen includes an elongated body that includes a hollow cannula portion that is open at both a proximal end and a distal end for placement at a specimen site to collect and permit aspiration of the specimen from the specimen site. The cannula portion has a plurality of side openings formed along a length of the cannula portion and proximate (near) the open distal end for providing entrances into a hollow interior of the cannula portion, wherein only one side opening is located in a single transverse plane taken across the cannula portion. In other words, the side openings are formed in the distalmost region of the cannula near the open distal end.

In another aspect of the present invention, the aspiration needle further includes a reinforcement element associated with each opening and extending at least substantially around a periphery of the opening. The reinforcement element can be in the form of a layer disposed on an outer surface of the cannula portion or it can be in the form of a grommet structure that is disposed on an outer surface of the cannula portion through the opening and to an inner surface of the cannula portion. Moreover, a reinforcement element can be formed opposite each opening (e.g., 180 degrees from the opening). Also, the reinforcement element can be constructed to include a means to assist in advancing the needle into bone, wherein the means includes a thread pattern (e.g., screw pattern) formed on and extending from the outer surface of the reinforcing element for engaging the bone as the needle is advanced into the bone.

According to another embodiment, an aspirating needle for collecting a specimen includes an elongated body that includes a hollow cannula portion that is open at both a proximal end and a distal end for placement at a specimen site to collect and permit aspiration of the specimen from the specimen site. The cannula portion has a raised thread pattern formed on a side wall of the lumen portion and longitudinally along a length of the cannula portion to assist in advancing the cannula portion into bone and to the specimen site. The raised thread pattern can be in the form of a helical screw pattern formed on the outer surface of the cannula portion.

According to yet another embodiment, an aspirating needle for collecting a specimen includes an elongated body that includes a substantially hollow cannula portion that is open at a proximal end and is closed at a pointed distal end for placement at a specimen site to collect and permit aspiration of the specimen from the specimen site. The cannula portion has a plurality of side openings formed along a length of the cannula portion and proximate the closed distal end for providing entrance ports into a hollow interior of the cannula portion, wherein only one side opening is located in a single transverse plane taken across the cannula portion.

Other features and advantages of the present invention will be apparent from the following detailed description when read in conjunction with the accompanying drawings.

### BRIEF DESCRIPTION OF THE DRAWING FIGURES

The foregoing and other features of the present invention will be more readily apparent from the following detailed description and drawing figures of illustrative embodiments of the invention in which:

FIG. 1 is a perspective view of an aspiration needle according to a first embodiment for use at a site that is aspirated to collect a sample, such as a fluid sample of bone marrow, and including at least one side entry port;

FIG. 2 is an enlarged cross-sectional view of the aspiration needle of FIG. 1 illustrating the side openings;

FIG. 3 is an enlarged partial cross-sectional view of an aspiration needle according to a second embodiment illustrating reinforcement of the side openings;

FIG. 4 is an enlarged partial cross-sectional view of an aspiration needle according to another embodiment illustrating reinforcement elements positioned opposite the side openings;

FIG. 5A is an enlarged partial side elevation view of an aspiration needle according to another embodiment illustrating side openings arranged in a helical manner, as well as a reinforcement element that has a helical shape and is offset from the side openings along the longitudinal axis;

FIG. 5B is an enlarged partial side elevation view of an aspiration needle according to another embodiment illustrating side openings arranged in a helical manner, as well as a reinforcement element that has a helical screw shape and is offset from the side openings along the longitudinal axis;

FIG. 5C is a cross-sectional view of the needle of FIG. 5B showing the helical screw reinforcement;

FIG. 6 is a partial cross-sectional view of an aspiration needle according to another embodiment; and

FIG. 7 is a cross-sectional view of an aspiration needle according to yet another embodiment.

### DETAILED DESCRIPTION OF PREFERRED EMBODIMENTS

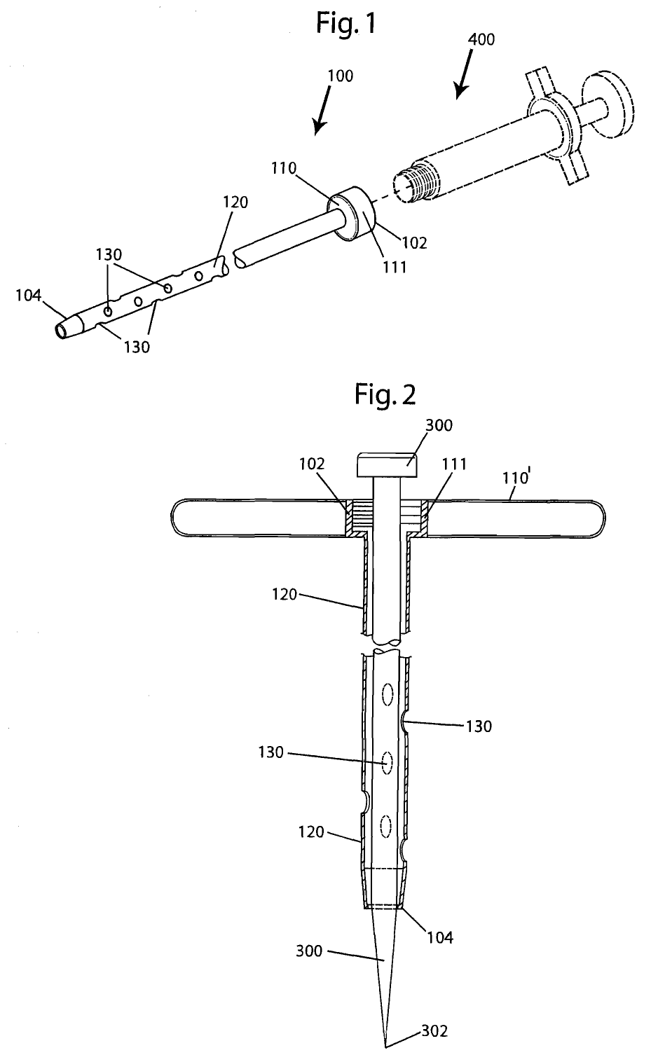

Referring now to FIG. 1, an aspiration needle 100 according to one exemplary embodiment is illustrated and is particularly suited for use at a target site, such as one associated with a bone marrow aspiration application. In other words, while the aspiration needle 100 is particularly suited for use in medical applications where aspiration of a local site takes place.

The aspiration needle 100 is constructed to overcome the deficiencies associated with conventional bone marrow aspiration needles and more specifically, is constructed to include additional means formed along the side wall of the needle and open to the interior of the needle for permitting entry and collection of the non-homogeneous aspirate sample.

The needle 100 has a first end 102 that is a proximal end and a second end 104 that is a distal end. The second end 104 (distal end) is a tip end for insertion into the cortical bone during the bone marrow collection procedure. The needle 100 is based on a lumen design in that the needle 100 that acts as a conduit for material to be withdrawn out of a bone marrow space into a syringe 400. The needle can thus include a body portion 110 that extends a predetermined distance from the proximal end 102 and a cannula portion 120 that extends from the body portion 110 to the distal end 104.

The body portion 110 has a feature that permits the needle 100 to be coupled to another component and therefore, can include a flange, cap, coupling member or the like. The first end 102 can serve as or be coupled to a hub and handle to facilitate the operators guiding the second end 104 safely into the appropriate position. The needle 100 shares a number of characteristics that are basic to most needles in that that the needle 100 is defined by an elongated structure (body and cannula) that is hollow in nature from one end 102 to the other end 104. The needle 100 can have any number of different shapes and for purpose of illustration only, the illustrated needle 100 has a circular cross-section; however, it will be appreciated that the cross-section of the needle 100 can be other shapes, including but not limited to square shaped, rectangular shaped, triangular shaped, etc.

The body portion 110 can take any number of different shapes and sizes and in the embodiment of FIG. 1, the body portion 110 is in the form of a hollow open ended annular structure (e.g., bowl shaped) that is defined by an annular wall 111 that extends between the open end 102 and another end which marks the interface between the cannula portion 120 and the body portion 110. In this present embodiment, the annular body portion 110 has a hollow interior that receives another object, such as syringe 400 for withdrawing the bone marrow sample or a stylet to assist in locating the distal end 104 of the needle 100 at the proper surgical site. In addition, the cannula portion 120 is in communication with the hollow interior of the body portion 110 to permit the bone marrow sample to flow from the cannula portion 120 to the interior of the body portion 110. The feature that is formed as part of the body portion 110 can be in the form of internal threads that are formed in the inside of the body portion 110. FIG. 2 shows an alternative type body portion 110′ in which an annular shaped flange extends radially outward to form a handle that the user can apply a force to as the needle 100 is being advanced into the specimen site.

The distal end 104 can also include a slight taper; however, in any event, the distal end 104 is an open end for collection of the specimen.

The cannula portion 120 is thus a generally unobstructed channel that extends from the first end 102 to the second end 104 and therefore, it permits material to be aspirated into the distal second end 104 and withdrawn to the first end 102 in a generally linear manner. At the first end 102, the aspirated material is delivered into the aspirating syringe device that is attached to the cannula portion 120 for storage thereof or is otherwise routed.

According to one aspect of the present invention, not only is the distal end 104 open but the needle 100 also includes at least one and preferably, a plurality of side aspiration entry ports (openings) 130 that permit the aspirate sample, e.g., bone marrow aspirate sample, to flow into the interior of the needle 100 (cannula portion 120) during operation of the aspiration procedure. The entry ports 130 are thus formed in the side wall of the needle 100 (cannula portion 120) and are thus formed along the longitudinal length thereof. Since the side openings 130 are intended to provide additional means besides the main opening at the distal end 102 to receive the aspirate sample into the interior of the needle 100, the side openings 130 are typically formed and located on the distal end portion of the cannula 120 since they should be formed in the section or portion of the cannula 120 that is placed at the specimen collection location.

It will be appreciated that the entry ports 130 are generally in the form of openings (apertures) that provide entry into the interior of the cannula 120. The openings 130 can take any number of different shapes and sizes that can be similar or different than the opening that is formed at the distal end 104. For example, the entry ports 130 can be in the form of circular or oval shaped openings that are arranged according to a predetermined pattern. It will be appreciated that any number of different patterns can be used when forming the openings 130 in the side wall of the needle 100. However, in order to provide a stable cannula 120, only one opening 130 should be formed in a single transverse plane that extends across the cannula 120. In other words, for any transverse plane cut transversely across the side wall of the cannula 120 and at right angle to a longitudinal axis of the cannula 120, there is only one opening 130 contained therein. This ensures that the structural rigidity of the cannula 120 is such that when the needle 100 is inserted through a resistant structure, such as bone, and into the bone marrow or other tissue at the site of the specimen acquisition, the cannula 120 will not bend along the transverse plane containing the opening 130. Since each opening 130 represents a weakened section of the cannula body, the placement of two or more openings 130 within the same transverse plane (cross-sectional cut across the cannula body at a right angle to the longitudinal axis of the cannula) would likely result in the cannula 120 being weakened along its longitudinal length at this location and thus, the odds of the cannula 120 snapping or bending at this region are increased.

It will be understood that the needle 100 is generally inserted with the stylet in place (through the needle 100) and that the stylet adds to the structural integrity of the needle assembly minimizing the risk of needle deformation or bending during insertion although not eliminating it. Also, the needle may be inadvertently or purposefully redirected after needle insertion when the stylet is no longer in place and in that situation, when the stylet's mechanical stability is lost, there might be increased tendencies for bending or deformation minimized by the placement of the entry points as described.

It will be appreciated that the openings 130 can be formed in any number of different patterns in the cannula 120 so long as two or more openings 130 are not contained in the same transverse plane as described above. In addition, it is preferred that the openings 130 be axially offset as well in that the openings are not formed along a single longitudinal axis along the length of the cannula 120 (however, this arrangement is possible). At least some of the openings 130 are thus preferably staggered or offset relative to one longitudinal axis that contains one opening 130. For example, the openings 130 can be formed in a helical (spiral) manner, as shown in FIG. 2, along the longitudinal axis of the cannula 120. Alternatively, the openings 130 can be in staggered pattern where every other opening 130 is formed along the same longitudinal axis; however, the intervening openings 130 are formed along a different longitudinal axis that is offset from the first longitudinal axis by a predetermined number of degrees. For example, the first set of openings 130 can be formed along a longitudinal axis that is at a 0 degree reference point, while the openings in the other longitudinal axis are formed about 90 degrees or 180 degrees from the 0 degree reference axis. It will be appreciated that once again no two openings 130 lie in the same transverse plane formed anywhere along the length of the cannula 120 at a right angle to the longitudinal axis of the cannula.

It will be appreciated that two or more openings 130 can lie within the same longitudinal axis of the cannula 120, as for example, in a helical pattern when the first opening 130 and a later opening 130, e.g., a third, fourth or fifth opening, etc., are in the same longitudinal axis.

The number of openings 130 is variable and can depend on a number of different parameters, including the size of the openings 130, the size of the distal tip opening at end 104, the intended application in terms of the makeup and location of the target specimen site, etc. Thus, while FIGS. 1 and 2 are shown with a prescribed number of openings 130, this is merely for illustrative purposes and does not limit the present invention in any way. It will further be appreciated that the needle 100 can include only a single opening 130 besides the main opening that is formed at the distal tip end 104.

The spacing between the openings 130 and the distance between the distal tip opening and the more distal side opening 130 are all variable and can come in any number of different arrangements so long as the structural integrity of the cannula 120 is not jeopardized by the positioning the openings 130 relative to each other and relative to the opening at the distal end 104.

The needle 100 of FIG. 1 incorporates a standard type needle design and a standard stylet 300 as shown in FIG. 1. As is well know, the stylet 300 is an elongated structure that has a complementary shape to the interior of the cannula 120 and has a size (e.g., outer diameter) that is slightly less than the size (inner diameter) of the cannula 120 to permit reception of the stylet 300 into the interior of the cannula 120 for providing temporary reinforcement of the cannula 120 along its longitudinal length. The stylet 300 has a pointed end 302 that protrudes through the opening at the distal tip end 104. The stylet remains in place during insertion of the aspiration needle 100 through the cortical bone until the distal tip opening and the side openings 130 are properly positioned within the bone marrow space or at another tissue site. The stylet would then be removed after this positioning.

Aspiration syringe 400 is placed at the proximal handle portion of the needle 100 and a negative pressure/suction force is applied through the cannula 120 for drawing (aspirating) the aspirate sample through the main distal tip opening and the side openings 130 and into the interior of the cannula 120 and then to a collector, such as a syringe attached to the body portion 110 for collection thereof. The stylet typically has a proximal knob or cap which allows the stylet to integrate and attach to the proximal portion of the cannula and/or the handle and keep its position within the cannula 120 during needle insertion. The locking mechanism between the stylet and handle can be any number of a variety of standard configurations that are used in aspiration needles or other similar types of needles.

It will thus be appreciated that the needle 100 of FIGS. 1 and 2 overcomes the deficiencies associated with the prior art by providing an aspiration needle 100 that has additional means for collecting the aspirate sample besides the traditional distal tip opening.

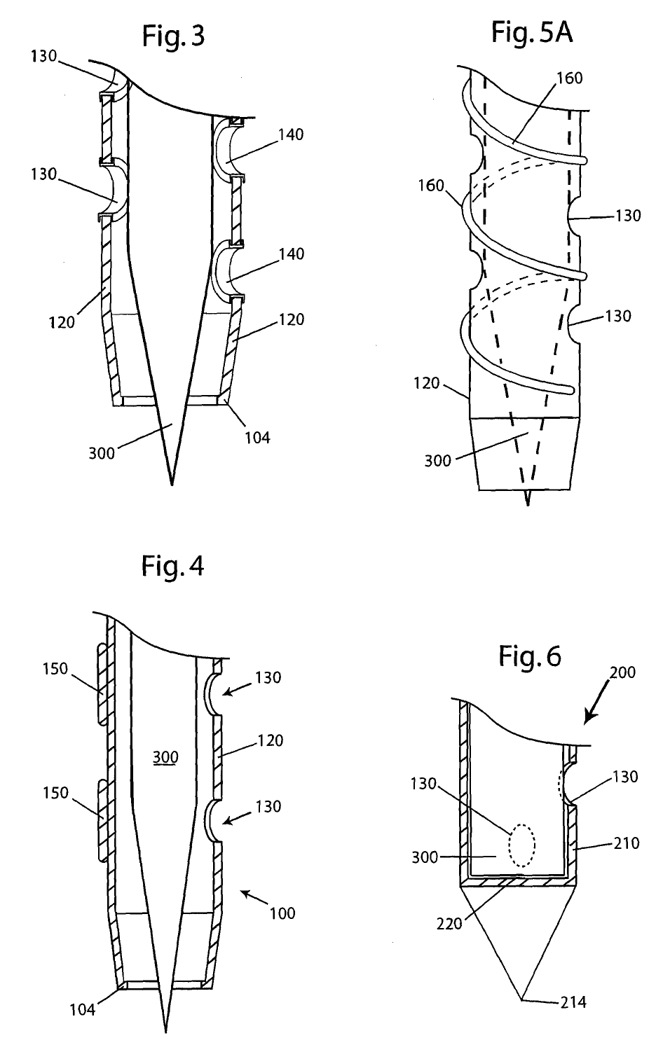

Now referring to FIG. 3 in which another aspect of the present invention is illustrated. As previously mentioned, the formation of openings (side ports) 130 in the side wall of the cannula 120, in theory, can destabilize the structural integrity of the needle 100 and the cannula 120 could bend or the distal end portion could even become separated and dislodged. By not having two or more openings 130 located in the same transverse plane, a minimization of the possible destabilization is achieved.

However, FIG. 3 illustrates the addition or incorporation of a reinforcement element 140 into the cannula 120 structure so as to further minimize the possibility of destabilization and to further strengthen the structural integrity of the needle 100. The reinforcement element 140 can be any number of different structures and be formed in any number of different locations along the cannula 120. For example and according to one embodiment, the reinforcement element 140 can be in the form of a ring or washer type structure that is formed around each opening 130 and is intended to increase the strength of the needle 100 in the region of the openings 130 to minimize cannula bending or collapse.

The reinforcement elements 140 can be formed only on the outer surface of the cannula 120 or they can be formed so that they extend at least into the opening 120 and can extend onto the inner surface of the cannula 120. In this manner, the reinforcement element 140 can have a grommet type structure that effectively reinforces the opening 130. In the illustrated embodiment, the reinforcement element 140 is in the form of a grommet type structure that improves and reinforces the cannula structure due to the bond between the reinforcement element and the cannula 120 around the periphery of the opening 130.

The reinforcement element 140 can be formed of any number of different materials so long as the material provides the desired structural reinforcement. For example, the reinforcement element 140 can be formed of a metal material that is bonded or otherwise securely attached to the cannula 120 and extends at least substantially around or completely around the opening 130.

These structures can be formed in a variety of different thicknesses and can be constructed so that they integrate and lock into the openings 130 in a number of different ways. The structures also can be integral to the cannula 120 itself as if the body of the cannula 120 were formed with increased thickness around the periphery of the openings 130 so as to reinforce the openings 130. In addition, a plating type operation can be performed for depositing the reinforcement material in a layer around the periphery of the opening 130. In this manner, the periphery can be built up with a reinforcement material. As with the other embodiments, the shape of the applied reinforcement material does not have to be identical to the shape of the opening 130. For example, the reinforcement element can be more of a square shaped layer formed around the circular opening 130.

Turning now to FIG. 4 in which yet another embodiment of the present invention is illustrated. In this embodiment, there is at least one side wall entry port (opening) 130 as well as the standard opening at the distal end 104. In this embodiment, for each or some side opening 130, there is a corresponding reinforcement element 150 that is located opposite the opening 130. In other words, 180 degrees opposite the opening 130 is a reinforcement element 150 that provides the desired structural reinforcement along the cannula body by increasing the thickness of the side wall that is directly opposite the location where the opening 130 is formed. The reinforcement element 150 can take any number of different shapes and sizes, with one being in the form of an elongated, longitudinal reinforcing strip or bar that extends a predetermined length of the cannula.

However, it will be appreciated that if the reinforcement element 150 surrounds the needle in a partial sleeve type configuration, then the reinforcement element can be from 90 degrees to 270 degrees from the centerline of the opening, with other configurations defined by a possibility of other angles. The most efficient structure would include material 180 degrees from the opening but support can be provided, as by having two element, one 120 degrees and one 200 degrees from the centerline of the opening, etc.

The reinforcement element 150 should have a length or other dimension that is at least equal to and preferably greater than the corresponding dimension of the opening 130. In other words, when the reinforcement element 150 is in the form of an elongate strip or bar, its length should be equal to or greater than a diameter of the opening 130.

The purpose of this linear strip of material is to minimize the tendency of the cannula 120 to bend at a location that is about 180 degrees opposite the opening 130. Without the structural enhancement, there would be a tendency for the proximal and distal aspects of the opening 130 to collapse resulting in the diameter of the side wall opening 130 decreasing and a bending of the needle 100. The reinforcement element 150 is designed to minimize the bending tendency and to keep the cannula 120 aligned longitudinally.

While one suitable material for the reinforcement element 150 is metal, it can likewise be formed of other materials that function in the intended manner. In addition, the material of the reinforcement element 150 should be capable of being bonded to the outer surface of the cannula 120.

It will also be appreciated that the reinforcement element 150 is not limited to having a strip or bar shape, but instead, can be formed in other shapes, such as a semi-circular pad or an oval pad or square pad that is positioned 180 degrees from the opening 130. However, as noted above, the pad can be at an angle other than 180 degrees.

In the case where there are two or more side openings 130, the reinforcement element 150 that corresponds to the openings 130 can be either a single unitary structure or it can be partitioned and be provided as single individual structure, one for each opening 130 as shown in FIG. 4.

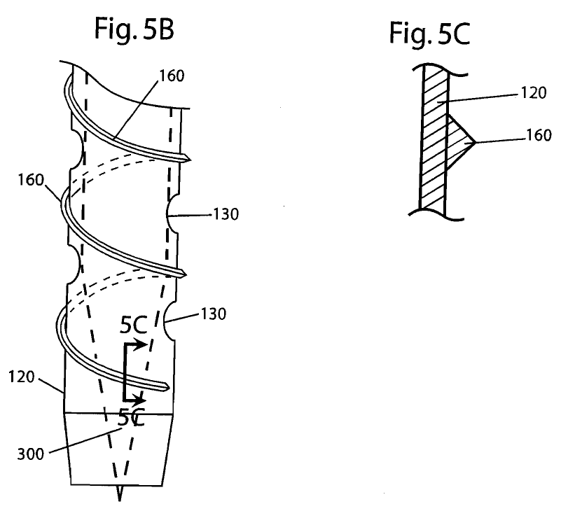

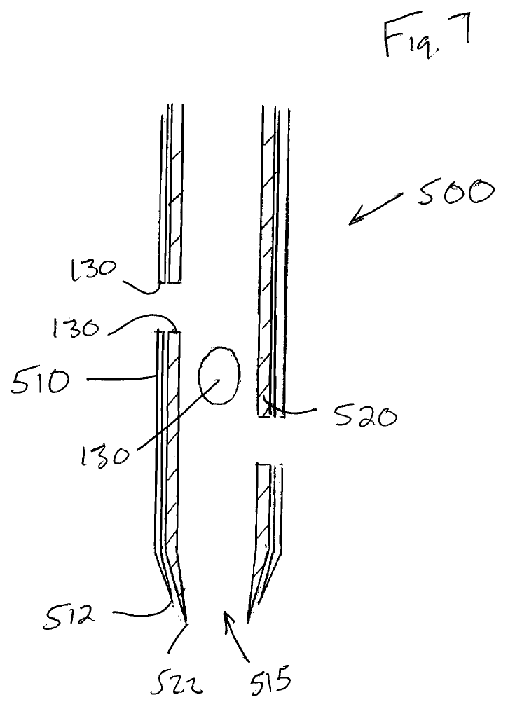

Now turning to FIGS. 5A-5C in which other aspects of the present invention is illustrated. In this embodiment, a curvilinear reinforcement element (stabilizing structure) 160 is provided and is designed to complement a cannula that has a plurality of openings 130 such that reinforcement and stabilization are provided 180 degrees opposite each opening 130. For example, when the openings 130 have a helical shape, the complementary reinforcement structure 160 likewise has a helical shape that is longitudinally offset from the helically arranged openings 130 so as to position a length of reinforcement material directly opposite the opening 130. Preferably and as mentioned above, the length of the reinforcement material is preferably substantially equal to or even greater than the dimension (diameter) of the opening 130. This type of structural or reinforcement element should increase the strength of the cannula 120 and minimize bending and collapse of the openings 130.

The helically shaped (spiral shaped) reinforcement element 160 can be integrated with the cannula 120 in a variety of different ways. The structure 160 can be formed on one or both of the outer surface and inner surface of the cannula 120 or it can even be integrated into the cannula wall itself. As with the previously described reinforcement elements, the reinforcement element 160 can be formed of any number of different materials, e.g., different metals, and can be securely attached to the cannula 120 using any number of different techniques, including bonding techniques, such as with an adhesive (glue) or by lasering, plating or some other technique for depositing material. It will be appreciated that other configurations for the reinforcement element 160 are possible, including multiple helixes that extend in different directions, etc.

Thus, both the openings 130 and the reinforcement element 160 assume a helical shape with one merely being longitudinally offset along the length of the cannula 120. The width of the reinforcement element 160 can vary depending upon a number of different parameters, including the size of the openings 130.

In yet another embodiment illustrated in FIGS. 5B and 5C, the reinforcement element 160 can be formed so that an outer surface thereof can provide a surface or lip on the helical shape to provide a means to assist rotation and advancement of the cannula 120 into the bone. More specifically, the outer surface of the reinforcement element 160 can be constructed to provide a helical screw type mechanism that provides the bone marrow aspiration needle 100 with a screw configuration that allows the cannula 120 to be advanced by rotating it into the bone which also helps secure it.

In other words, the outer surface of the reinforcement can include a screw type thread that allows the cannula 120 to be easily advanced into the bone as for example by applying a torque force to the handle 110 of the needle 100. The screw type thread means can be formed as by any number of different ways, including roughening the outer surface of the helical reinforcement 160 or providing an etched pattern thereof, such as cross threads, that cause the reinforcement 160 to act as a thread as the cannula 120 is being advanced. The thread (reinforcement 160) can be integral to the outer wall of the cannula if the helical valley is grooved into the wall resulting in a corresponding helical radial projection.

It will further be appreciated that the thread means 160 can be provided on the outer surface of a cannula that includes only one side wall opening 130 or the thread means 160 can be formed on the outer surface of a cannula that includes no side wall openings 130 and the only means for entering the interior is through the main cannula opening at the distal end 104.

In addition, a helical shape for the thread means 160 is merely exemplary and other shapes are equally possible so long as they perform the intended function of assisting in the cannula 120 being advanced into the bone.

Now referring to FIG. 6 in which another aspect of the present invention is illustrated. In this embodiment, an aspiration needle 200 is provided and while similar to the needle 100, the needle 200 has a number of differences, including that it is a closed tip needle in that a distal end 214 of a cannula 210 is not open as in the needle 100 but instead, the distal end 214 is closed. In order to facilitate the advancement of the distal end 214, the closed end preferably includes a solid pointed distal tip as opposed to the tapered distal end (frustoconical shape) of the needle 100 that terminates in the main opening into the interior of the needle 100.

In order to provide access into the interior of the cannula 210, the needle 200 does include one or more side entry points (openings 130) that provide access into the interior of the cannula 210 and are formed in the distal end region so that they are located where the bone marrow aspirate sample can easily be aspirated into and collected in the interior of the cannula 210.

It will be appreciated that this type of design can be more advantageous if the needle 200 is passed through the bone marrow space and the tip (distal end 214) comes to lodge against the opposite cortical bone. In that case, the distal tip opening in a conventional needle would be obstructed and serve little purpose, especially, since this distal tip opening is the main and usually only entrance into the interior of the cannula. In contrast and according to this embodiment, the distal tip 214 is configured as a solid structural element that can stabilize the needle 200 when it penetrates into the contralaterial cortical bone table. In that situation, the openings 130 will be more distal to the contralateral bone table and thus, are situated in the bone marrow space.

In yet another aspect, since the distal end 214 is not open, the stylet can not simply pass through the hollow interior of the cannula and out the distal tip opening but instead, proximate the solid tip at the distal end 214, a floor 220 is formed that represents the closed end of the cannula 410. In this embodiment, the distal tip of the stylet 300 abuts against the floor 220 adjacent to the closed distal tip 214. The stylet functions in the traditional sense in that it remains in the needle 200 during the process of penetrating the bone and it provides structural support and reinforcement to the needle 200 since it is contained within the hollow cannula 210, thereby effectively filling up the hollow interior with a solid object and thereby adding robustness to the entire needle 200 and permitting it to be more easily advanced into the bone without worrying about the needle 200 bending or otherwise collapsing due to its hollow nature. The stylet 300 would then be removed once the aspiration needle 200 is positioned properly in the bone. Unlike conventional stylets, once this stylet is removed, its removal opens up all of the side openings 130 to facilitate the aspiration procedure, without any opening at the distal tip itself through which the sample is collected.

In yet another embodiment illustrated in FIG. 7, an aspiration needle 500 is provided that has a number of features that are identical or similar to the features described with reference to the previous embodiments. For example and for sake of simplicity, the handle portion or the needle 500 is not illustrated and the stylet is also not illustrated.

In this embodiment, the aspiration needle 500 is of a double walled structure in that the needle 500 is formed of a first cannula structure 510 and a second cannula structure 520 that is received within the first cannula structure 510. The first and second cannula structures 510, 520 complement one another and thus, have complementary shapes and dimensions to permit the first cannula structure 510 to be in the form of an outer sleeve that surrounds the second cannula structure 520 that represents an inner cannula 520. Each of the outer sleeve 510 and the inner cannula 520 has a distal tip end 512, 522, respectively. The distal tips 512, 522 can terminate at the same location or the distal tip 522 of the inner cannula 520 can extend slightly beyond the distal tip 512 of the outer sleeve 510.

The distal tips 512, 522 are formed at the ends of tapered sections of the outer sleeve 510 and inner cannula 520 and are pointed, sharpened ends to permit piercing of tissue to assist in delivering the needle 500 to the target specimen site. In this embodiment, each of the distal tips 512, 522 includes a distal opening 515 to permit collection of the specimen.

It will be appreciated that the dimensions and/or material make-up of the outer sleeve 510 and the inner cannula 520 can be different from one another. More specifically, the thickness of the outer sleeve 510 and the inner cannula 520 can be different or they can be the same. Also, the shapes/constructions of the outer sleeve 510 and the inner cannula 520 can be different in that the outer sleeve 510 can be formed without a tapered distal tip region but still surrounds a substantial length of the inner cannula 520 so as to provide added reinforcement and strengthening of the needle 500 structure. In other words, the outer sleeve 510 can extend to the distal tip 522 of the inner cannula 520 or it can terminate prior thereto and thus, the length of the outer sleeve 510 can be different from the length of the inner cannula 520 or it can be the same.

In one embodiment, the outer sleeve 510 can be formed of a non-metal material, while the inner cannula 520 is formed of a metal material, such as stainless steel. For example, the outer sleeve 510 can be formed of a plastic material or a composite material and in one embodiment, the outer sleeve is formed of a plastic or composite coating that is applied to the outer surface of the inner cannula 520. In particular, the coating material can be provided as a liquid (e.g., molten substance) that is applied as a layer (coating) to the outer surface of the inner cannula 520 as by dipping the inner cannula 520 within a batch of molten material (e.g., polymeric material) that is restricted from flowing within the interior of the inner cannula 520. After cooling, the material hardens to form the outer sleeve 510.

In another embodiment, the outer sleeve 510 and the inner cannula 520 can be separate cannula structures that are attached to one another along their lengths using conventional techniques, such as bonding the outer sleeve 510 and the inner cannula 520 together. The two can be bonded together using any number of different techniques, including the use of an adhesive material, such as glue, or by providing a laser bond between the two structures.

Regardless of the type of structures, the needle 500 is formed so that each of the outer sleeve 510 and the inner cannula 520 is perforated to include one or more openings 130 that provide entry into the interior of the inner cannula 520. The openings 130 can take any number of different shapes and sizes that can be similar or different than a distal opening that is formed at the distal ends 512, 522. For example, the openings 130 can be in the form of circular or oval shaped openings that are arranged according to a predetermined pattern. It will be appreciated that any number of different patterns can be used when forming the openings 130 in the side wall of the needle 500 through both the outer sleeve 520 and the inner cannula 520. However, in order to provide a stable and robust needle 500, only one opening 130 should be formed in a single transverse plane that extends across the inner cannula 520. In other words, for any transverse plane cut transversely across the side wall of the cannula 520 and at right angle to a longitudinal axis of the cannula 520, there is only one opening 130 contained therein. This ensures that the structural rigidity of the cannula 520 is such that when the needle 500 is inserted through a resistant structure, such as bone, and into the bone marrow or other tissue at the site of the specimen acquisition, the cannula 520 will not bend along the transverse plane containing the opening 130.

It will be understood that the needle 500 is generally inserted with the stylet in place (through the needle 100) and that the stylet adds to the structural integrity of the needle assembly minimizing the risk of needle deformation or bending during insertion although not eliminating it. Also, the needle may be inadvertently or purposefully redirected after needle insertion when the stylet is no longer in place and in that situation, when the stylet's mechanical stability is lost, there might be increased tendencies for bending or deformation minimized by the placement of the entry points as described.

It will be appreciated that the openings 130 can be formed in any number of different patterns in the cannula 520 and outer sleeve 510 so long as two or more openings 130 of the inner cannula 520 or outer sleeve 510 are not contained in the same transverse plane as described above. In addition, it is preferred that the openings 130 be axially offset as well in that the openings are not formed along a single longitudinal axis along the length of the cannula 520 and outer sleeve 510 (however, this arrangement is possible). At least some of the openings 130 are thus preferably staggered or offset relative to one longitudinal axis that contains one opening 130. For example, the openings 130 can be formed in a helical (spiral) manner, as shown in FIG. 7, along the longitudinal axis of the cannula 520 and outer sleeve 510. Alternatively, the openings 130 can be in staggered pattern where every other opening 130 is formed along the same longitudinal axis; however, the intervening openings 130 are formed along a different longitudinal axis that is offset from the first longitudinal axis by a predetermined number of degrees. For example, the first set of openings 130 can be formed along a longitudinal axis that is at a 0 degree reference point, while the openings in the other longitudinal axis are formed about 90 degrees or 180 degrees from the 0 degree reference axis. It will be appreciated that once again no two openings 130 lie in the same transverse plane formed anywhere along the length of the cannula 520 and outer sleeve 510 at a right angle to the longitudinal axis of the cannula and outer sleeve.

It will be appreciated that two or more openings 130 can lie within the same longitudinal axis of the cannula 520 and outer sleeve 510, as for example, in a helical pattern when the first opening 130 and a later opening 130, e.g., a third, fourth or fifth opening, etc., are in the same longitudinal axis.

The number of openings 130 is variable and can depend on a number of different parameters, including the size of the openings 130, the size of the distal tip opening at ends 512, 522, the intended application in terms of the makeup and location of the target specimen site, etc.

The spacing between the openings 130 and the distance between the distal tip opening and the more distal side opening 130 are all variable and can come in any number of different arrangements so long as the structural integrity of the cannula 520 is not jeopardized by the positioning the openings 130 relative to each other and relative to the opening at the distal end 104.

It will also be appreciated that the dimensions of the openings 130 formed in the inner cannula 520 can be different than the dimensions of the openings 130 formed in the outer sleeve 510. FIG. 7 shows the openings 130 having the same dimensions to permit direct access to the interior of the inner cannula 520. In another embodiment, the openings 130 of the inner cannula 520 have greater dimensions (or smaller) than the dimensions of the openings 130 in the outer sleeve 510.

In yet another aspect, the openings 130 formed in the outer sleeve 510 can be at least partially axially offset from the openings 130 formed in the inner cannula 520. It is contemplated that the outer sleeve 510 can be rotatably mounted or coupled to the inner cannula 520 such that it can be locked into a desired position with respect to the inner cannula 520. In this manner, rotation of the outer sleeve 510 with respect to the inner cannula 520 causes either an increased overlap between the openings 130 formed in the inner cannula 520 and the outer sleeve 510 or a decreased overlap between the openings 130. Depending upon the particular surgical operation, either increased or decreased access to the interior of the inner cannula 520 may be desired.

The provision of outer sleeve 510 to the needle 500 and more particularly, to the inner cannula 520 provides a number of advantages including that is it an easier and more economic way of manufacturing a reinforced needle structure. It results in a reinforced needle since the width of the needle 500 is increased by providing the outer sleeve 510 over the inner cannula 520. The outer sleeve 510 does not necessarily have to be a thick structure since a relatively thin layer of a strong material, such as a composite or reinforced fiber structure (synthetic polymeric materials), can serve the purpose without increasing substantially the width of the needle.

The outer sleeve 520 provides an effective yet relatively easy way of providing increased reinforcement to the needle 500 while still permitting the needle 500 to include one or more and preferably a plurality of openings 130 that provide direct access to the interior of the inner cannula 520.

The needle 500 can be of the type that has an open distal end as shown in FIG. 7 or it can be of the type that has a closed distal end as shown in FIG. 6 with respect to an earlier embodiment.

While exemplary drawings and specific embodiments of the present invention have been described and illustrated, it is to be understood that the scope of the present invention is not to be limited to the particular embodiments discussed. Thus, the embodiments shall be regarded as illustrative rather than restrictive, and it should be understood that variations may be made in those embodiments by workers skilled in the art without departing from the scope of the present invention as set forth in the claims that follow, and equivalents thereof. In addition, the features of the different claims set forth below may be combined in various ways in further accordance with the present invention.

What is claimed is:

1. An aspirating needle for collecting a specimen comprising:
an elongated hollow body that includes a cannula portion that is open at both a proximal end and a distal end for placement at a specimen site to collect and permit aspiration of the specimen from the specimen site, the cannula portion has a plurality of side openings formed along a length of the cannula portion and proximate the open distal end for providing entrances into a hollow interior of the cannula portion, wherein only one side opening is located in a single transverse plane taken across the cannula portion and at a right angle to a longitudinal axis of the cannula portion.

2. The aspiration needle of claim 1, wherein the plurality of side openings are formed according to a helically shaped pattern along a length of the cannula portion.

3. The aspiration needle of claim 1, wherein the plurality of side openings are arranged in a staggered pattern with each side opening being formed in a different longitudinal axis defined along a length of the cannula portion.

4. The aspiration needle of claim 1, wherein the plurality of side openings are formed such that an angle between any pair of side openings is different from an angle between another pair of side openings.

5. The aspiration needle of claim 1, wherein at least one of a shape and a size of each side opening differs from a shape and size, respectively, of the opening defined at the distal end of the cannula portion.

6. The aspiration needle of claim 1, wherein each opening is substantially circular shaped.

7. The aspiration needle of claim 1, further comprising a reinforcement element associated with each opening and extending at least around substantially around a periphery of the opening.

8. The aspiration needle of claim 7, wherein the reinforcement element comprises a layer disposed on an outer surface of the cannula portion.

9. The aspiration needle of claim 7, wherein the reinforcement element comprises a grommet structure that is disposed on an outer surface of the cannula portion through the opening and to an inner surface of the cannula portion.

10. The aspiration needle of claim 7, wherein the reinforcement element is formed of a metal material that is different from a material of the cannula portion.

11. The aspiration needle of claim 1, further comprising a reinforcement element formed opposite the opening.

12. The aspiration needle of claim 11, wherein the reinforcement element is formed 180 degrees from the opening.

13. The aspiration needle of claim 11, wherein the reinforcement element comprises a layer formed on an outer surface of the cannula portion.

14. The aspiration needle of claim 11, wherein the reinforcement layer comprises a linear segment of material.

15. The aspiration needle of claim 11, wherein the reinforcement element comprises a plurality of pads formed of a reinforcing material, with one pad being formed on an opposite side of the side wall of the cannula portion.

16. The aspiration needle of claim 11, wherein the side openings are formed in a helical pattern and the reinforcement element has a helical pattern that is offset longitudinally from the helically shaped openings such that reinforcing material is located opposite each opening.

17. The aspiration needle of claim 11, wherein the reinforcing material comprises a section of the cannula portion that has increased thickness relative to surrounding portions.

18. The aspiration needle of claim 16, wherein the reinforcing element is constructed to provide a means to assist advancement the needle into bone.

19. The aspiration needle of claim 18, wherein the means comprises a thread pattern formed on and extending from the outer surface of the reinforcing element for engaging the bone as the needle is advanced into the bone.

20. An aspirating needle for collecting a specimen comprising:
an elongated body that includes a hollow cannula portion that is open at both a proximal end and a distal end for placement at a specimen site to collect and permit aspiration of the specimen from the specimen site, the cannula portion has a raised thread pattern formed on a side wall of the cannula portion and longitudinally along a length of the cannula portion to assist in advancing the cannula portion into bone and to the specimen site.

21. The aspirating needle of claim 20, wherein the raised thread pattern is in the form of a helical screw pattern formed on the outer surface of the cannula portion.

22. The aspirating needle of claim 20, further comprising at least one opening formed in the side wall to form an entrance into an interior of the cannula portion to permit the specimen to be received and collected within the interior.

23. An aspirating needle for collecting a specimen comprising:
an elongated body that includes a substantially hollow cannula portion that is open at a proximal end and is closed at a pointed distal end for placement at a specimen site to collect and permit aspiration of the specimen from the specimen site, the cannula portion has a plurality of side openings formed along a length of the cannula portion and proximate the closed distal end for providing entrances into a hollow interior of the cannula portion, wherein only one side opening is located in a single transverse plane taken across the cannula portion at a right angle to a longitudinal axis of the cannula portion.

24. The aspirating needle of claim 23, wherein the plurality of side openings are formed according to a helically shaped pattern along a length of the cannula portion.

25. The aspiration needle of claim 23, wherein the plurality of side openings are arranged in a staggered pattern with each side opening being formed in a different longitudinal axis defined along a length of the cannula portion.

26. The aspiration needle of claim 1, further comprising a reinforcement element associated with each opening and extending at least substantially around a periphery of the opening.

27. The aspiration needle of claim 26, wherein the reinforcement element comprises a grommet structure that is disposed on an outer surface of the cannula portion through the opening and to an inner surface of the cannula portion.

28. The aspiration needle of claim 23, further comprising a reinforcement element that is formed opposite the opening.

29. The aspiration needle of claim 28, wherein the reinforcement element is formed 180 degrees from the opening.

30. The aspiration needle of claim 28, wherein the reinforcement layer comprises one of a linear segment of material and a plurality of pads formed of a reinforcing material, with one pad being formed on an opposite side of the side wall of the cannula portion.

31. The aspiration needle of claim 28, wherein the side openings are formed in a helical pattern and the reinforcement element has a helical pattern that is offset longitudinally from the helically shaped openings such that reinforcing material is located opposite each opening.

32. The aspiration needle of claim 28, wherein the reinforcing element is constructed to provide a means to assist in advancing the needle into bone.

33. The aspiration needle of claim 32, wherein the means comprises a thread pattern formed on and extending from the outer surface of the reinforcing element for engaging the bone as the needle is advanced into the bone.
